(19) United States
(12) Patent Application Publication
(10) Pub. No.: US 2008/0045774 A1
(43) Pub. Date: Feb. 21, 2008

(54) METHODS FOR TREATING DRUG ADDICTION, OBESITY AND EPILEPSY

(76) Inventor: Shi Peng Song, Shanghai (CN)

Correspondence Address: MATTHIAS SCHOLL, 14781 MEMORIAL DRIVE, SUITE 1319, HOUSTON, TX, 77079, US

(21) Appl. No.: 11/462,718

(22) Filed: Aug. 7, 2006

(51) Int. Cl. A61N 2/00

(52) U.S. Cl. 600/9

(57) ABSTRACT

This invention provides methods for treating diseases related to cerebral disturbances, such as drug addiction, obesity and epilepsy. A method for treating diseases related to cerebral disturbances comprises: (a) imaging the cerebrum of a patient, (b) identifying a locus of cerebral disturbance, and (c) irradiating the locus of cerebral disturbance with a therapeutically effective dose of electromagnetic radiation.

### BACKGROUND OF THE INVENTION

1. Field of the Invention

This invention relates to methods for treating diseases related to cerebral disturbances, and especially to methods for treating drug addiction, obesity and epilepsy.

2. Description of Related Art

Existing methods for treating drug addiction, obesity and epilepsy are drug-based. Known drugs, however, can only reduce symptoms of these diseases but generally do not result in a complete regression of the diseases. In addition drugs may cause many adverse reactions.

### BRIEF SUMMARY OF THE INVENTION

This invention provides, among other, methods for treating diseases related to cerebral disturbances.

In one embodiment, a method for treating diseases related to cerebral disturbances in a patient in the need of such treatment comprises: (a) imaging the cerebrum of a patient, (b) identifying a locus of the cerebral disturbance, and (c) irradiating said locus with a therapeutically effective dose of electromagnetic radiation.

In a class of this embodiment, the imaging of the cerebrum is performed at least once during an episode of a cerebral disturbance and at least one additional time in the absence of an episode of the cerebral disturbance, and the identifying of the locus of the cerebral disturbance is done by comparing images obtained from imaging the cerebrum during an episode of cerebral disturbance and images obtained from imaging the cerebrum in the absence of an episode of cerebral disturbance.

In a class of this embodiment, the disease (related to a cerebral disturbance) is selected from the group of drug addiction, obesity and epilepsy.

In a class of this embodiment, the therapeutically effective dose of electromagnetic radiation adsorbed by the patient is between about 1 and about 30 Gy, and preferably is between about 2 and about 10 Gy. In a subclass of this embodiment, one or more doses amounting to a total of between about 2 and about 10 Gy are administered on one day. In a subclass of this embodiment, one or more doses amounting to a total of between about 2 and about 10 Gy are administered over a period of several days, weeks, months, and/or years. In a subclass of this embodiment, one or more doses each of between about 2 and about 10 Gy are administered over a period of several days, weeks, months, and/or years.

In a class of this embodiment, converging beams of radiation are used to irradiate a locus of the cerebral disturbance.

### DETAILED DESCRIPTION OF THE INVENTION

Definitions

The term “disease,” as used herein, refers to an interruption, cessation, or disorder of body functions, systems, or organs, which are related to cerebral disturbances.

The term “related to cerebral disturbances” as used in connection with the term “disease” refers to the fact that there is some relationship (association and/or causation) between the disease and the cerebral disturbances. The term “disease,” as used herein, is intended to include conditions, symptoms, syndromes, abnormalities, etc., which are related to cerebral disturbances.

The term “cerebral disturbances” refers to any disorders or abnormalities of the cerebrum which can be diagnosed using imaging techniques, such as CT, SPECT, PET, etc.

The term “treating” or “treatment” of a disease in a patient includes: (1) preventing the disease from occurring in a mammal which may be predisposed to the disease but does not yet experience or display symptoms of the disease, (2) inhibiting the disease, i.e., arresting its development, or (3) relieving symptoms of the disease, i.e., causing regression of the disease, including a complete regression of the disease.

An “effective amount” or “inhibiting dosage” of, e.g., radiation, with respect to the subject method of treatment, refers to an amount of radiation which, when absorbed as part of a desired dosage regimen brings about treatment of the disease according to clinically acceptable standards for the disorder to be treated. The term “therapeutically effective amount” refers to the amount which, when adsorbed by an animal for treating a disease, is sufficient to effect such treatment of the disease.

Radiation absorbed dose is often measured and stated in the older units called rad; the System International of units (SI system) for radiation measurement uses the units “gray” (Gy) for absorbed dose of radiation. 1 Gy corresponds to 100 rad.

A “patient” or “subject” to be treated by the subject method can mean either a human or non-human animal.

The term “CT” refers to X-ray Computed Tomography. It is a diagnostic imaging procedure that uses a combination of x-rays and computer technology to produce cross-sectional images (often called slices), both horizontally and vertically, of the body. A CT scan shows detailed images of any part of the body, including the bones, muscles, fat, and organs. CT scans are more detailed than standard X-rays. In computed tomography, the x-ray beam moves in a circle around the body. This allows many different views of the same organ or structure. The x-ray information is sent to a computer that interprets the x-ray data and displays it in a two-dimensional (2D) form on a monitor.

The term “SPECT” refers to Single Photon Emission Computed Tomography. It is a nuclear medicine tomographic imaging technique using gamma rays. It is very similar to conventional nuclear medicine planar imaging using a gamma camera. However, it is able to provide true 3D information. This information is typically presented as cross-sectional slices through the patient, but can be freely reformatted or manipulated as required.

The term “PET” refers to Positron Emission Tomography. It is a tomographical imaging technique that uses ligands or substrates labeled with positron-emitting radioisotopes to image physiological processes or molecular events.

The terms “radioactive source,” “radiation source,” and “source,” as used herein, refer to sources of electromagnetic radiation useful for treating diseases in a patient in need of such treatment. Contemplated radiation sources for use in methods according to the invention described herein include but are not limited to X-ray sources, incl. linear accelerators, and may include without limitation other sources, such as, e.g., neutron sources, gamma ray sources, nuclear particle sources, ion sources, electron sources, proton sources, microwave sources, and radio frequency sources. Radiation sources, as used herein, also include radioactive isotopes.

The term “medical imaging” refers to the various methods used to show detailed images of any part of the body, including the bones, muscles, fat, and organs. The term “medical imaging” is intended to include without limitation: SPET (Singe Photon Emission Tomography), SPECT (Single Photon Emission Computed Tomography), PET (Positron Emission Tomography), CT (X-ray Computed Tomography), and a combination of these techniques, such as, e.g., PET/CT or SPET/CT, etc.

The terms “locus” and “target,” as used herein, with respect to the cerebrum of a patient refer to an area of the cerebrum that (1) has been identified through scanning of the cerebrum by means of medical imaging or via any other methods of identification employing, e.g., electrophysical devices such as, e.g., EEG (electroencephalogram), and/or (2) is irradiated so as to treat diseases related to cerebral disturbances.

Methods of the present invention eliminate completely diseases such as drug addiction, obesity and epilepsy. While not wishing to be bound by any theory, it is believed that the irradiation of the disturbances-causing locus in the cerebrum alters, aborts and/or prevents neuronal excitement responsible for causing diseases related to cerebral disturbances.

### EXAMPLE 1

Method for Treating Drug Addiction

Employing medical imaging techniques such as PET/CT or SPET/CT, cerebral scans of subjects are obtained: (1) before occurrence of symptoms of drug addiction, (2) during the commencement of symptoms of drug addiction, (3) during the peak phase of symptoms of drug addiction, and (4) during the diminishing phase of symptoms of drug addiction, respectively. Then, by comparing and analyzing the obtained images of the cerebrum, high-activity metabolic regions and low-activity metabolic regions are identified. Specifically, by comparing and analyzing the obtained images of the cerebrum, neuronal excitement centers for drug addiction are identified. The neuronal excitement centers are then irradiated with converging x-rays at a dose of 10 Gy. Upon irradiation, patients were no longer addicted to drugs.

### EXAMPLE 2

Method for Treating Obesity and/or Reducing Weight.

Employing medical imaging techniques such as PET/CT or SPET/CT, cerebral scans of subjects are obtained: (1) when subjects were fairly hungry and in strong appetite and (2) when subjects were full, satisfied and comfortable. Then, by comparing and analyzing the obtained images of the cerebrum, high-activity metabolic regions and low-activity metabolic regions are identified. The high-activity regions correspond to the “appetite centre” (also referred to as the “hunger centre”). The appetite centers are/were then irradiated with converging x-rays at a dose of 5 Gy. Upon irradiation, patients exhibited loss of appetite and lost weight to normal levels.

### EXAMPLE 3

Method for Treating Epilepsy.

Employing medical imaging techniques such as PET/CT or SPET/CT, cerebral scans of subjects suffering from epilepsy are obtained with subjects wearing a relocatable head frame: (1) prior to epileptic attacks, and (2) during the course of an epileptic attack (e.g., which was purposefully induced). Then, by comparing and analyzing the obtained images of the cerebrum, high-activity metabolic regions and low-activity metabolic regions are identified. The high-activity regions showing up as bright dots in the images correspond to the epilepsy-causing centers. The epilepsy-causing centers are/were then irradiated with an inhibiting dosage of converging x-rays at a dose of 20 Gy. Upon irradiation, patients no longer suffer/suffered from epilepsy.

Whereas, particular embodiments of the invention have been described above for purposes of illustration, it will be evident to those skilled in the art that numerous variations of the details may be made without departing from the invention as defined in the appended claims.

What is claimed is:

1. A method for treating a disease related to a cerebral disturbance in a patient in the need of such treatment comprising:
(a) imaging the cerebrum of the patient,
(b) identifying a locus of the cerebral disturbance, and
(c) irradiating said locus with a therapeutically effective dose of electromagnetic radiation.

2. The method of claim 1, wherein
said imaging of the cerebrum is performed at least once during an episode of a cerebral disturbance and at least one additional time in the absence of an episode of the cerebral disturbance, and
said identifying of the locus of the cerebral disturbance is done by comparing images obtained from imaging the cerebrum during an episode of cerebral disturbance and images obtained from imaging the cerebrum in the absence of an episode of cerebral disturbance.

3. The method of claim 1, wherein said disease is selected from the group of drug addiction, obesity and epilepsy.

4. The method of claim 2, wherein said disease is selected from the group of drug addiction, obesity and epilepsy.

5. The method of claim 1, wherein said therapeutically effective dose of electromagnetic radiation is between about 1 and about 30 Gy.

6. The method of claim 2, wherein said therapeutically effective dose of electromagnetic radiation is between about 1 and about 30 Gy.

7. The method of claim 1, wherein said therapeutically effective dose of electromagnetic radiation is between about 2 and about 10 Gy.

8. The method of claim 2, wherein said therapeutically effective dose of electromagnetic radiation is between about 2 and about 10 Gy.
